(19) United States
(12) Patent Application Publication
(10) Pub. No.: US 2003/0113353 A1
(43) Pub. Date: Jun. 19, 2003

(54) Fast hydrating dispersible biopolymer

(76) Inventors: Sophie Vaslin, Saint-Cloud (FR); Arnaud Lyothier, Aubervilliers (FR)

Correspondence Address: Kevin McVeigh, Intellectual Property Department, Rhodia Inc, 259 Prospect Plains Road CN 7500, Cranbury, NJ, 08512-7500, US

(21) Appl. No.: 10/203,853

(22) PCT Filed: Feb. 15, 2001
(86) PCT No.: PCT/FR01/00454

(30) Foreign Application Priority Data
Feb. 18, 2000 (FR) 00/02037

(51) Int. Cl. A61K 9/14; A01N 25/00; C09K 3/00

(52) U.S. Cl. 424/401; 504/358; 507/213

(57) ABSTRACT

The invention concerns a dispersible and fast hydrating biopolymer in dry form, characterized in that at least 75 wt. % of said biopolymer particles have a diameter ranging between 60 and 250 microns and a mean diameter (d50) ranging between 100 and 200 microns. The invention also concerns the use of such a biopolymer as thickening or viscosity, emulsifying and/or stabilizing agent in industrial, food, cosmetic and pharmaceutical formulations.

The present invention relates to dispersible and fast-hydrating biopolymers in dry form and to their use as thickeners, emulsifiers and/or stabilizers for the preparation of industrial formulations (such as, for example, the construction, paints, paper, textiles, plant-protection, water-treatment and petroleum industries), food, cosmetic, agrochemical and pharmaceutical formulations.

Biopolymers of high molecular weight, which are polysaccharides in the context of the present invention, are increasingly used in many industrial applications for their thickening, viscosity-modifying, emulsifying and/or stabilizing properties in media, especially aqueous media. Thus, xanthan gum, on account of its exceptional rheological properties, is used in fields as varied as construction, paints, paper, textiles, cosmetics, food, agriculture, water treatment and oil drilling and recovery.

For many applications, when the biopolymer is in dry form, it is necessary to place it in aqueous solution. However, the major drawback of these powders is their difficulty in dispersing and hydrating quickly in a given aqueous medium. Often, the dispersion and/or hydration of these biopolymers results in the formation of lumps, which are harmful to the functionality of the medium.

In the case of a thickening biopolymer such as xanthan, the improvement in terms of dispersion and hydration without the formation of lumps allows in particular better control of the viscosity of the reaction medium.

For this reason, it would be advantageous to develop a biopolymer, in dry form, in particular of xanthan gum type, which can be easily dispersed and quickly hydrated in aqueous media while at the same time reducing or even eliminating the formation of lumps.

In practice, one of the solutions recommended to avoid the formation of lumps while at the same time maintaining good dispersibility and hydratability of the powders consists in modifying the polymer powder by the so-called “granulation” method. This method consists in aggregating the biopolymer powder in slightly humid medium (“fluid bed” technique) in order to obtain granules with an average size of between 300 and 1000 microns. The major drawback of this method lies in its high implementation cost.

Another solution consists in supplementing the biopolymer with a compound of surfactant type. However, the addition of such compounds does not correspond with the current trend which is to reduce the number of additives added to a given formulation, in particular in the food sector.

The aim of the present invention is to propose a biopolymer powder which, in aqueous media, without the need to add an additive and/or vigorous stirring means, gives an improved dispersion and fast hydration while at the same time reducing or even eliminating the formation of lumps.

To this end, a subject of the invention is a dispersible and fast-hydrating biopolymer, in dry form, at least 75% by weight of the particles of which have a diameter of between 60 and 250 microns and a mean diameter (d₅₀) of between 100 and 250 microns.

Preferably, at least 75% by weight of the biopolymer particles have a diameter of between 100 and 200 microns and a mean diameter (d₆₀) of between 100 and 200 microns.

The judicious selection of the particle size distribution of the biopolymers according to the invention makes it possible both to control the dispersion of the powder and to promote its hydration while at the same time substantially reducing and usually entirely eliminating the formation of lumps.

The biopolymers of the invention also have the advantage of being dust-free and easy to pour.

Other advantages and characteristics of the present invention will emerge more clearly on reading the description, the examples and the figures which follow.

In the present description, the notation d₅₀ represents the particle size distribution such that 50% by volume of the particles are less than or equal to the said size. For example, a d₅₀ of 100 microns means that 50% by volume of the particles are less than or equal to 100 microns in size.

The particle size is determined by laser spectroscopy with a machine such as a “Coulter LS 230” machine in dry-route configuration.

In the context of the present invention, the term “biopolymer” more particularly denotes polysaccharides of high molecular weight, usually greater than 1×10⁶ g/mol (measured by gel permeation) and which consists of glucose, mannose, galactose, rhamnose, glucuronic acid, mannuronic acid and guluronic acid units, optionally with acetate and pyruvate derivatives. Their particular structure and their properties are disclosed, for example, in the book Industrial Gums—Whistler—2nd edition—chapters XXI-XXIII (1973).

These biopolymers are advantageously produced by aerobic culturing of microorganisms in an aqueous nutrient medium.

Many microorganisms such as bacteria, yeasts, fungi and algae are capable of producing these biopolymers. Mention may be made, inter alia, of:

bacteria belonging to the genus Xanthomonas and more particularly to the species described in Bergey's Manual of Determinative Bacteriology (8th edition—1974—Williams N. Wilkins Co. Baltimore) such as Xanthomonas begoniae, Xanthomonas campestris, Xanthomonas carotae, Xanthomonas hederae, Xanthomonas incanae, Xanthomonas malvacearum, Xanthomonas papavericola, Xanthomonas phaseoli, Xanthomonas pisi, Xanthomonas vasculorum, Xanthomonas vesicatoria, Xanthomonas vitians and Xanthomonas pelargonii;

bacteria belonging to the genus Arthrobacter and more particularly the species Arthrobacter stabilis and Arthrobacter viscosus;

bacteria belonging to the genus Erwinina;

bacteria belonging to the genus Azotobacter and more particularly the species Azotobacter indicus;

bacteria belonging to the genus Agrobacterium and more particularly the species Agrobacterium radiobacter, Agrobacterium rhizogenes and Agrobacterium tumefaciens;

bacteria belonging to the genus Alcaligenes and more particularly Alcaligenes faecalis;

bacteria belonging to the genus Pseudomonas and more particularly Pseudomonas methanica;

bacteria belonging to the genus Corynebacterium;

bacteria belonging to the genus Bacillus and more particularly Bacillus polymyxa;

fungi belonging to the genus Sclerotium and more particularly to the species Sclerotium glucanicum, Sclerotium rolfsii or Plectania occidentalis;

fungi belonging to the genus Aspergillus and more particularly to the species Aspergillus itaconicus and Aspergillus terreus;

yeasts belonging to the genus Hansenula, such as the species Hansenula capsulata.

In one preferred embodiment of the invention, the microorganism is a bacterium of the genus Xanthomonas, more particularly the species Xanthomonas campestris, and the biopolymer is xanthan gum.

The biopolymers according to the invention may be obtained by any process for controlling the particle size distribution of a powder. By way of example, mention may be made of screening.

As indicated above, the biopolymers according to the present invention disperse easily in aqueous media and do not require high-shear means. The dispersibility of the biopolymer powder is evaluated by counting the lumps formed when the biopolymer is placed in aqueous solution. The greater the number of lumps formed, the poorer the dispersibility.

The hydration speed may be determined, for example, by measuring the viscosity developed over time under gentle stirring conditions. As a guide, gentle stirring may correspond to stirring with a deflocculating paddle at a speed which may be between 400 and 600 rpm.

These characteristics make it possible to incorporate the biopolymers according to the invention into many formulations, as thickeners, viscosity modifiers, emulsifiers and/or stabilizers.

The invention also covers the use of a biopolymer as defined above as a thickener, viscosity modifier, emulsifier and/or stabilizer in:

formulations for the drilling for and assisted recovery of petroleum and for water treatment;

formulations for paper, construction and textiles;

food, cosmetics, agrochemical and pharmaceutical formulations;

formulations for industrial and household cleaning; as well as the said formulations containing such a biopolymer.

The examples given below are given by way of non-limiting illustration of the present invention.

### KEY TO THE FIGURES

FIG. 1 represents the particle size distribution of the samples: products A, B and C, obtained using a Coulter LS230 granulometer in dry-route configuration.

FIG. 2 represents the hydration speed of samples A, B and C, in distilled water. This speed is determined by measuring the viscosity developed over time under the conditions of Example 1—moisturization test in distilled water.

FIG. 3 represents the hydration speed of samples A, B and C, in a medium containing 40% sucrose. This speed is determined by measuring the viscosity developed over time under the conditions of Example 2.

### EXAMPLES

### Example 1

Preparation of the Samples

Product A: Product A is a standard commercial xanthan gum powder sold under the name Rhodigel® by the company Rhodia.

Product B: Product B is according to the invention. It is a xanthan gum with a particle size of between 180 μm and greater than 125 μm. This product is obtained by two simultaneous screenings: one screening through a screen with a mesh size of 180 μm and one screening through a 125 μm screen.

Product C: Product C is a xanthan gum powder which has undergone a granulation by so-called “fluid bed” technique according to Example 1 of patent FR-A-2 600 267.

TABLE 1

| Sample | Product A | Product B | Product C |
|---|---|---|---|
| % of particles between | 68 | 98 | 38 |
| 60 and 250 microns | | | |
| % of particles between | 48 | 79 | 18 |
| 100 and 200 microns | | | |
| d₅₀ microns | 149.6 | 180.8 | 177.1 |

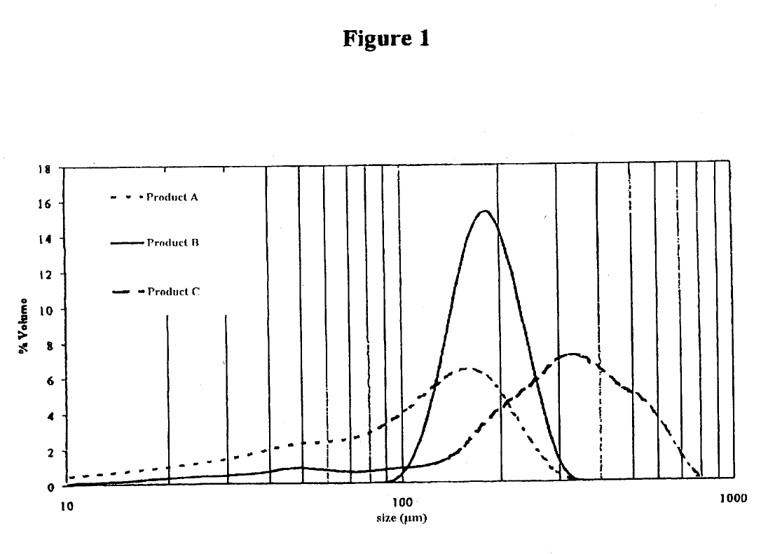

FIG. 1 represents the particle size distribution of samples A, B and C.

Dispersibility Test

The dispersion properties of a hydrocolloid may be evaluated by a test for counting the number of lumps present in a solution after dispersion.

Solutions of xanthan gum at 0.3% in distilled water are prepared. This dissolution is carried out on an amount of 500 ml of distilled water, in a low-line 1000 ml beaker, with a deflocculating paddle 65 mm in diameter, at a speed of 400 rpm for 15 min.

The said solution is filtered through a screen with a 1 mm mesh size and the number of lumps present on the screen after filtration is recorded.

The results are summarized in Table 2.

TABLE 2

| Product | Number of lumps |
|---|---|
| Product A | 55 |
| Product B | 0 |
| Product C | 0 |

A product is considered as

“VERY GOOD” if the number of lumps is less than 5;

“GOOD” if the number of lumps is less than 10;

“POOR” if the number of lumps is greater than 10.

Hydration Test

This test makes it possible to evaluate the hydration speed of a biopolymer powder in aqueous media.

0.3% of xanthan gum powder (products A, B and C) is dissolved in distilled water. This dissolution is carried out on an amount of 500 ml of distilled water, in a 1000 ml low-line beaker, with a deflocculating paddle 65 mm in diameter, at a speed of 400 rpm for 15 min. The viscosity of the said solution is measured at four different moments, using a Brookfield LVT viscometer, spindle No. 2, set at 12 rpm. These four moments are at 1, 2, 5 and 15 minutes.

The viscosity values are given in Table 3 below:

TABLE 3

| | Viscosity | | Viscosity | Viscosity | Viscosity |
|---|---|---|---|---|---|
| Product | at 1 min | | at 2 min | at 5 min | at 15 min |
| Product A | 20 | mPa · s | 100 mPa · s | 332 mPa · s | 605 mPa · s |
| Product B | 238 | mPa · s | 692 mPa · s | 747 mPa · s | 742 mPa · s |
| Product C | 137 | mPa · s | 712 mPa · s | 687 mPa · s | 675 mPa · s |

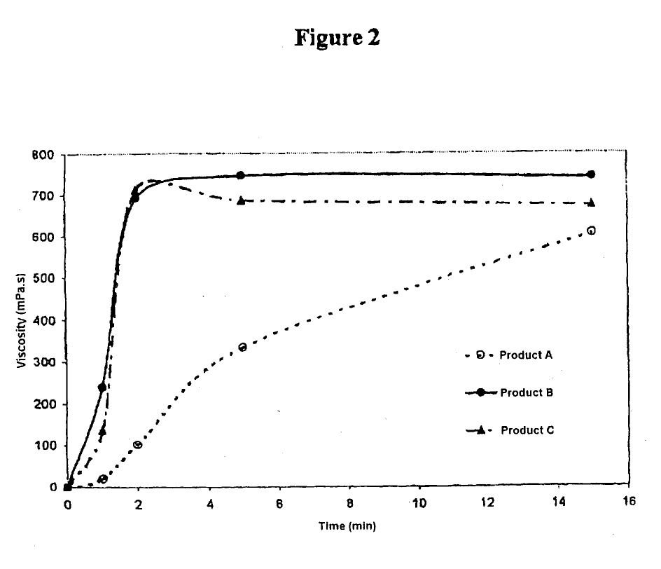

FIG. 2 represents the hydration speed of samples A, B and C, in distilled water. This speed is determined by measuring the viscosity developed over time.

### Example 2

Hydration Test in a 40% Sucrose Solution

This test makes it possible to evaluate the hydration speed of a biopolymer powder in sugar-containing aqueous media.

0.3% of xanthan gum powder (products A, B and C) is dissolved in a solution containing 40% sucrose. This dissolution is carried out on an amount of 500 g of 40% sucrose solution, in a 1000 ml low-line beaker, with a deflocculating paddle 65 mm in diameter, at a speed of 400 rpm for 30 minutes. The viscosity of the said solution is measured at three different moments, using a Brookfield LVT viscometer, spindle No. 2, set at 12 rpm. These three moments are at 5, 15 and 30 minutes.

The viscosity values are given in Table 4.

TABLE 4

| | Viscosity at | | Viscosity at | Viscosity at |
|---|---|---|---|---|
| Product | 5 min | | 15 min | 30 min |
| Product A | 870 | mPa · s | 1140 mPa · s | 1280 mPa · s |
| Product B | 970 | mPa · s | 1470 mPa · s | 1480 mPa · s |
| Product C | 1075 | mPa · s | 1430 mPa · s | 1410 mPa · s |

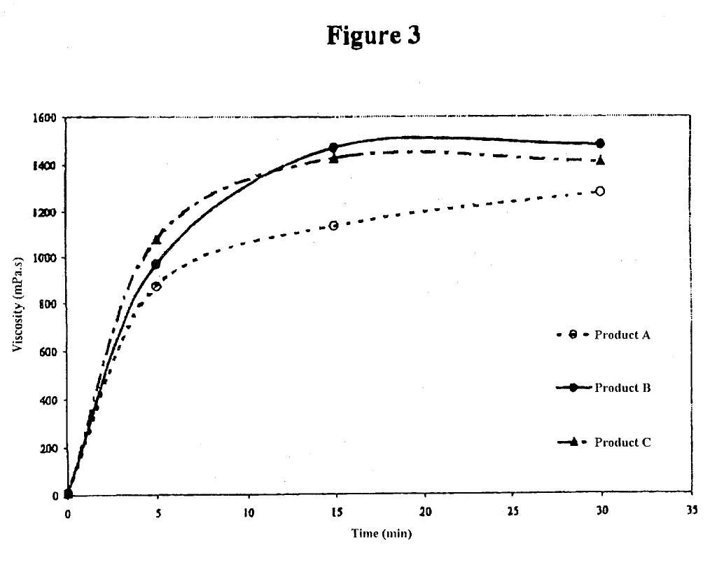

FIG. 3 represents the hydration speed of samples A, B and C, in a 40% sucrose medium. This speed is determined by measuring the viscosity developed over time.

1. Dispersible and fast-hydrating biopolymer-in dry form, characterized in that at least 75% by weight of the particles of the said biopolymer have a diameter of between 60 and 250 microns and a mean diameter (d₅₀) of between 100 and 250 microns.

2. Biopolymer according to claim 1, characterized in that at least 75% by weight of the particles of the said biopolymer have a diameter of between 100 and 200 microns and a mean diameter (d₅₀) of between 100 and 200 microns.

3. Biopolymer according to either of claims 1 and 2, characterized in that the said biopolymer is a xanthan gum.

4. Use of a biopolymer according to any one of claims 1 to 3 as a thickener, viscosity modifier, emulsifier and/or stabilizer.

5. Use of a biopolymer according to any one of claims 1 to 3 as a thickener, viscosity modifier, emulsifier and/or stabilizer in formulations for the drilling for and the assisted recovery of petroleum and for water treatment.

6. Use of a biopolymer according to any one of claims 1 to 3 as a thickener, viscosity modifier, emulsifier and/or stabilizer in food, cosmetics, pharmaceutical and agrochemical formulations.

7. Use of a biopolymer according to any one of claims 1 to 3 as a thickener, viscosity modifier, emulsifier and/or stabilizer in formulations for industrial and household cleaning.

8. Use of a biopolymer according to any one of claims 1 to 3 as a thickener, viscosity modifier, emulsifier and/or stabilizer in formulations for paper, construction and textiles.

9. Formulation for drilling for and assisted recovery of petroleum, for water treatment, food, cosmetics, pharmaceuticals or agrochemicals, for industrial and household cleaning or for paper, construction or textiles, using a biopolymer according to any one of claims 1 to 3, as a thickener, viscosity modifier, emulsifier and/or stabilizer.
